(19) United States
(12) Patent Application Publication
(10) Pub. No.: US 2024/0332610 A1
(43) Pub. Date: Oct. 3, 2024

(54) MOLECULAR CRYSTAL, ELECTROCHEMICAL DEVICE, AND METHOD FOR PRODUCING MOLECULAR CRYSTAL

(71) Applicant: TOYOTA JIDOSHA KABUSIKI KAISHA, Toyota-shi Aichi-ken (JP)

(72) Inventors: Kazuya Takeuchi, Sunto-gun Shizuoka-ken (JP); Satoru Nakashima, Susono-shi Shizuoka-ken (JP)

(73) Assignee: TOYOTA JIDOSHA KABUSIKI KAISHA, Toyota-shi Aichi-ken (JP)

(21) Appl. No.: 18/616,313

(22) Filed: Mar. 26, 2024

(30) Foreign Application Priority Data
Mar. 31, 2023 (JP) 2023-058447

(51) Int. Cl. H01M 10/0564; C07D 285/15; H01M 10/052

(52) U.S. Cl. CPC H01M 10/0564; C07D 285/15; H01M 10/052; H01M 2300/0065

(57) ABSTRACT

A molecular crystal with lithium ion conductivity is disclosed. The molecular crystal of the present disclosure includes sulfolane and a lithium salt represented by the following chemical formula and the molar ratio of said sulfolane to said lithium salt is 2.0 or more and less than 3.0.

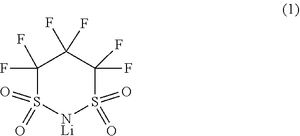

### CROSS-REFERENCE TO RELATED APPLICATIONS

This application claims priority to Japanese Patent Application No. 2023-058447 filed Mar. 31, 2023, the entire contents of which are herein incorporated by reference.

### FIELD

The present application discloses a molecular crystal, an electrochemical device and a method of manufacturing a molecular crystal.

### BACKGROUND

PTL 1 discloses a molecular crystal solid electrolyte comprising an electron-donating sulfur-based organic compound and a lithium salt.

### CITATION LIST

### Patent Literature

[PTL 1] JP 2013-214510 A

### SUMMARY

### Technical Problem

Conventional molecular crystals have room for improvement in terms of ionic conductivity.

### Solution to Problem

As a technique for solving the above problem, the present application discloses the following plurality of aspects.

### <Aspect 1>

A molecular crystal, comprising a sulfolane and a lithium salt represented by the following chemical formula (1), wherein
a molar ratio of the sulfolane to the lithium salt is 2.0 or more and 3.1 or less.

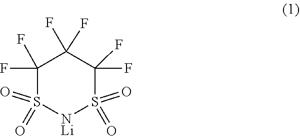

### <Aspect 2>

A molecular crystal of Aspect 1, having a hexagonal structure.

### <Aspect 3>

A molecular crystal of Aspect 1 or 2, wherein
the molar ratio of the sulfolane to the lithium salt is 2.0 or more and less than 3.0.

### <Aspect 4>

An electrochemical device, comprising: an ionic conductor, wherein
the ionic conductor has a molecular crystal of any of Aspects 1 to 3.

### <Aspect 5>

A method for producing a molecular crystal, the method comprising:
mixing sulfolane and a lithium salt represented by the following chemical formula (1) to obtain a solution containing the sulfolane and the lithium salt and having a molar ratio of the sulfolane to the lithium salt of 2.0 or more and 3.1 or less, and
cooling the solution to obtain the molecular crystal.

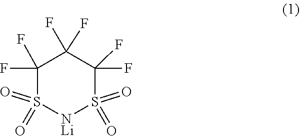

### Effects

The molecular crystal of the present disclosure has ionic conductivity.

### BRIEF DESCRIPTION OF DRAWINGS

FIG. 1 schematically shows an example of the configuration of an electrochemical device.

FIG. 2 shows X-ray diffraction patterns of the solid samples of Examples 1 to 5 and Comparative Examples 2, 5, and 8.

### DESCRIPTION OF EMBODIMENTS

Hereinafter, embodiments of the technology of the present disclosure will be described, but the technology of the present disclosure is not limited to the embodiments described below.

### 1. Molecular Crystal

A molecular crystal according to an embodiment includes sulfolane and a lithium salt represented by the following chemical formula (1). The molar ratio of the sulfolane to the lithium salt is 2.0 or more and 3.1 or less.

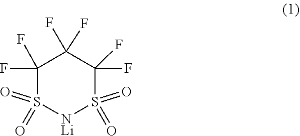

### 1.1 Sulfolane

The molecular crystal according to the present embodiment includes sulfolane. Sulfolane is a kind of electron-donating sulfur-based organic compound and is represented by the following chemical formula (2). In the molecular crystal, the sulfolane represented by the following chemical formula (2) may or may not be chemically bonded to the lithium salt represented by the above chemical formula (1). The molecular crystal according to an embodiment may have a hexagonal structure depending on the orientation of each molecule while maintaining the molecular structure of sulfolane and the anionic molecular structure of the lithium salt.

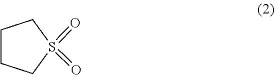

### 1.2 Lithium Salt

The molecular crystal according to the present embodiment includes a predetermined lithium salt. The lithium salt is lithium 1,1,2,2,3,3-hexafluoropropane-1,3-disulfonimide represented by the above chemical formula (1). According to the findings of the present inventor, the ionic conductivity of the above sulfolane alone and the above lithium salt alone is difficult to measure because of high resistance. As in the molecular crystal according to the present embodiment, when the above sulfolane and the above lithium salt are combined, ion conductivity is exhibited.

### 1.3 Molar Ratio

In the molecular crystal according to the present embodiment, the molar ratio of the above sulfolane to the above lithium salt (sulfolane/lithium salt) is 2.0 or more and 3.1 or less. In other words, the molecular crystal according to the present embodiment contains 2.0 mol or more and 3.1 mol or less of sulfolane with respect to 1 mol of lithium salt. According to the findings of the present inventor, if the molar ratio is too high, liquid is partially generated and an appropriate form as a molecular crystal cannot be maintained. Further, if the molar ratio is too low, ion conductivity tends to decrease. In the molecular crystal according to the present embodiment, when the molar ratio is 2.0 or more and 3.1 or less, ion conductivity is specifically improved. The molar ratio may be 2.0 or more and 3.0 or less, 2.0 or more and less than 3.0, or 2.0 or more and 2.9 or less. Note that the molar ratio of sulfolane to lithium salt (sulfolane/lithium salt) in the molecular crystal can be specified by various analyzers.

### 1.4 Crystal Structure

The molecular crystal according to the present embodiment may have a specific crystal structure by including the above sulfolane and the above lithium salt. For example, the molecular crystal according to the present embodiment may have a hexagonal structure. In molecular crystals having a hexagonal structure, it is considered that Li layer exists in x-y plane direction of the hexagonal structure, and that there are substantially no components in x-y plane that interfere with the diffusion of Li, whereby Li easily diffuses. As a result, it is considered that ionic conductivity is expressed. The molecular crystal according to the present embodiment may be made of a hexagonal structure single phase or may have other crystal structures together with a hexagonal structure. It is considered that more ion conductivity is expressed when it is composed of a hexagonal structure single phase.

### 1.5 Others

### 1.5.1 Morphology at Room Temperature

The molecular crystal according to the present embodiment may be solid at room temperature (20° C.) under an air atmosphere. In other words, the molecular crystal according to the present embodiment may be one having ion conductivity in a solid state.

### 1.5.2 Other Ingredients

The molecular crystal according to the present embodiment may be composed of only the above-mentioned sulfolane and lithium salt or may contain some component in addition to these. In the molecular crystal concerning this embodiment, for example, the sum total of the above-mentioned sulfolane and lithium salt may occupy 50 mass % or more and 100 mass % or less, 60 mass % or more and 100 mass % or less, 70 mass % or more and 100 mass % or less, 80 mass % or more and 100 mass % or less, 90 mass % or more and 100 mass % or less, 95 mass % or more and 100 mass % or less, or 99 mass % or more and 100 mass % or less.

### 1.5.3 Use

The molecular crystal according to the present embodiment has ion conductivity and can be applied to various applications. The molecular crystal according to the present embodiment may be used as a solid electrolyte.

### 2. Electrochemical Device

The molecular crystal according to the present embodiment has ion conductivity and is applicable as a constituent material of various electrochemical devices. That is, an electrochemical device according to an embodiment includes an ion conductor, wherein the ion conductor has a molecular crystal of the present disclosure. Hereinafter, a battery as an electrochemical device will be exemplified, but the molecular crystal of the present disclosure is applicable to an electrochemical device other than a battery.

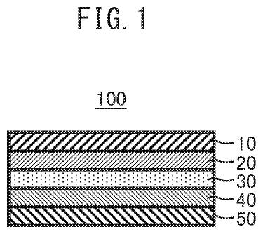

As shown in FIG. 1, a battery 100 according to an embodiment includes a positive electrode active material layer 20, an electrolyte layer 30, and a negative electrode active material layer 40. At least one of the positive electrode active material layer 20, the electrolyte layer 30, and the negative electrode active material layer 40 includes the molecular crystal of the present disclosure. In other words, in the battery 100, the molecular crystal of the present disclosure may function as a solid electrolyte. In the battery 100, a configuration other than a molecular crystal as a solid electrolyte is the same as in the prior art. For example, a configuration as described in JP-A-2014-186937 and JP-A-2021-068556 can be adopted. As shown in FIG. 1, the battery 100 may include a positive electrode current collector 10 electrically connected to the positive electrode active material layer 20 described above, or a negative electrode current collector 50 electrically connected to the negative electrode active material layer 40 described above. As the configuration of the current collector, any known configuration may be employed. In addition, the battery 100 may have a general configuration as a battery in addition to the above-described configuration. For example, a tab, a terminal, or the like. In addition, the battery 100 may be one in which each of the above-described configurations is housed inside an exterior body. Any of the exterior bodies known as an exterior body of a battery can be employed as the exterior body. In addition, a plurality of batteries 100 may be optionally electrically connected and optionally superimposed to form a battery assembly. The shapes of the battery 100 may include, for example, coin-shaped, laminated, cylindrical, and square-shaped, and the like. The battery 100 may be a lithium-ion battery. The battery 100 may be a primary battery or a secondary battery. The battery 100 may be manufactured by, for example, forming each of the above-described layers in a dry or wet manner, or the like.

### 3. Method for Producing Molecular Crystals

The molecular crystals of the present disclosure may be produced, for example, by the following method. That is, a method of manufacturing a molecular crystal according to an embodiment comprises:
Step S1: mixing a sulfolane and a lithium salt represented by the above chemical formula (1) to obtain a solution containing the sulfolane and the lithium salt and having a molar ratio of the sulfolane to the lithium salt of 2.0 or more and 3.1 or less, and
Step S2: cooling the solution to obtain the molecular crystal.

### 3.1 Step S1

In step S1, sulfolane and a lithium salt represented by the above chemical formula (1) are mixed to obtain a solution containing the sulfolane and the lithium salt and having a molar ratio of the sulfolane to the lithium salt of 2.0 or more and 3.1 or less. For example, after obtaining a melt of sulfolane, a predetermined amount of lithium salt is added thereto and mixed with heating, whereby the lithium salt is dissolved in the melt to obtain a solution. There is no particular limitation on the mixing conditions, and any conditions may be used as long as it is possible to appropriately mix sulfolane and lithium salt to obtain a solution. In mixing, sulfolane and a lithium salt may be stirred. For example, a lithium salt may be added to a melt of sulfolane to dissolve the lithium salt, and then stirred for 1 minutes or more and 10 hours or less, whereby the sulfolane and the lithium salt may be uniformly mixed. There is no particular limitation on the stirring technique.

### 3.2 Step S2

In step S2, the solution obtained by step S1 is cooled to obtain the molecular crystal. There is no particular limitation on the cooling conditions. For example, the solution may be allowed to cool to room temperature under an air atmosphere.

### EXAMPLES

As described above, one embodiment of the technology of the present disclosure has been described, but the technology of the present disclosure can be variously modified other than the above embodiments without departing from the gist thereof. Hereinafter, the technique of the present disclosure will be described in further detail with reference to Examples, but the technique of the present disclosure is not limited to the following Examples.

### 1. Preparation of Evaluation Samples

### 1.1 Comparative Example 1

A lithium salt represented by the following chemical formula (1) and a sulfolane represented by the following chemical formula (2) were weighed so that a molar ratio of said sulfolane to said lithium salt was 3.5. After the sulfolane was heated to form a melt, a lithium salt was added to the melt, and the lithium salt was dissolved by stirring with warming. After stirring for 2 hours, by allowing to cool to room temperature in air, to obtain an evaluation sample according to Comparative Example 1. The evaluation sample obtained was a mixture of solid and liquid at room temperature (20° C.) under an air atmosphere.

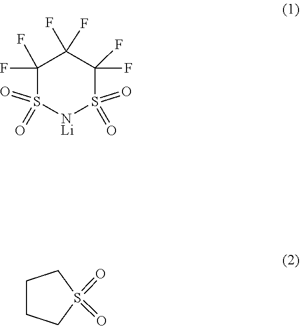

### 1.2 Example 1

An evaluation sample according to Example 1 was obtained in the same manner as in Comparative Example 1, except that the molar ratio of said sulfolane to said lithium salt was 3.1. The obtained evaluation sample was a molecular crystal which was solid at room temperature (20° C.) under an air atmosphere and contained sulfolane and a lithium salt.

### 1.3 Example 2

An evaluation sample according to Example 2 was obtained in the same manner as in Comparative Example 1, except that the molar ratio of said sulfolane to said lithium salt was 3.0. The obtained evaluation sample was a molecular crystal which was solid at room temperature (20° C.) under an air atmosphere and contained sulfolane and a lithium salt.

### 1.4 Example 3

An evaluation sample according to Example 3 was obtained in the same manner as in Comparative Example 1, except that the molar ratio of said sulfolane to said lithium salt was 2.9. The obtained evaluation sample was a molecular crystal which was solid at room temperature (20° C.) under an air atmosphere and contained sulfolane and a lithium salt.

### 1.5 Example 4

An evaluation sample according to Example 4 was obtained in the same manner as in Comparative Example 1, except that the molar ratio of said sulfolane to said lithium salt was 2.5. The obtained evaluation sample was a molecular crystal which was solid at room temperature (20° C.) under an air atmosphere and contained sulfolane and a lithium salt.

### 1.6 Example 5

An evaluation sample according to Example 5 was obtained in the same manner as in Comparative Example 1, except that the molar ratio of said sulfolane to said lithium salt was 2.0. The obtained evaluation sample was a molecular crystal which was solid at room temperature (20° C.) under an air atmosphere and contained sulfolane and a lithium salt.

### 1.7 Comparative Example 2

An evaluation sample according to Comparative Example 2 was obtained in the same manner as in Comparative Example 1, except that the molar ratio of said sulfolane to said lithium salt was 1.5. The obtained evaluation sample was a molecular crystal which was solid at room temperature (20° C.) under an air atmosphere and contained sulfolane and a lithium salt.

### 1.8 Comparative Example 3

LiPF₆ as a lithium salt and a sulfolane represented by the above chemical formula (2) were weighed so that the molar ratio of said sulfolane to said lithium salt was 4.0. After the sulfolane was heated to form a melt, a lithium salt was added to the melt, and the lithium salt was dissolved by stirring with warming. After stirring for 2 hours, by allowing to cool to room temperature in air, to obtain an evaluation sample according to Comparative Example 3. The evaluation sample obtained was a mixture of solid and liquid at room temperature (20° C.) under an air atmosphere.

### 1.9 Comparative Example 4

Lithium bis (fluorosulfonyl) imide (LiFSI (also referred to as lithium bis (fluorosulfonyl) amide, LiFSA)) as a lithium salt and a sulfolane represented by the above chemical formula (2) were weighed so that the molar ratio of said sulfolane to said lithium salt was 1.5. After the sulfolane was heated to form a melt, a lithium salt was added to the melt, and the lithium salt was dissolved by stirring with warming. After stirring for 2 hours, by allowing to cool to room temperature to obtain an evaluation sample according to Comparative Example 4. The evaluation sample obtained was a mixture of solid and liquid at room temperature (20° C.) under an air atmosphere.

### 1.10 Comparative Example 5

An evaluation sample according to Comparative Example 5 was obtained in the same manner as in Comparative Example 4, except that the molar ratio of said sulfolane to said lithium salt was 1.0. The obtained evaluation sample was a molecular crystal which was solid at room temperature (20° C.) under an air atmosphere and contained sulfolane and a lithium salt.

### 1.11 Comparative Example 6

Lithium bis (trifluoromethanesulfonyl) imide (LiFSI (also referred as lithium bis (trifluoromethanesulfonyl) amide, LiTFSA)) as a lithium salt and a sulfolane represented by the above chemical formula (2) were weighed so that the molar ratio of said sulfolane to said lithium salt was 2.0. After the sulfolane was heated to form a melt, a lithium salt was added to the melt, and the lithium salt was dissolved by stirring with warming. After stirring for 2 hours, by allowing to cool to room temperature in air, to obtain an evaluation sample according to Comparative Example 6. The obtained evaluation sample was a liquid at room temperature (20° C.) under an air atmosphere.

### 1.12 Comparative Example 7

An evaluation sample according to Comparative Example 7 was obtained in the same manner as in Comparative Example 6, except that the molar ratio of said sulfolane to said lithium salt was 1.5. The evaluation sample obtained was a mixture of solid and liquid at room temperature (20° C.) under an air atmosphere.

### 1.13 Comparative Example 8

An evaluation sample according to Comparative Example 8 was obtained in the same manner as in Comparative Example 6, except that the molar ratio of said sulfolane to said lithium salt was 1.0. The obtained evaluation sample was a molecular crystal which was solid at room temperature (20° C.) under an air atmosphere and contained sulfolane and a lithium salt.

### 1.14 Comparative Example 9

The lithium salt represented by the above chemical formula (1) and the ethyl methyl sulfone represented by the following chemical formula (3) were weighed so that the molar ratio of the ethyl methyl sulfone to the lithium salt was 1.0. Ethyl methyl sulfone was heated to form a melt, and then a lithium salt was added thereto, and the mixture was stirred with warming to dissolve the lithium salt. After stirring for 2 hours, by allowing to cool to room temperature in air, to obtain an evaluation sample according to Comparative Example 9. The obtained evaluation sample was a liquid at room temperature (20° C.) under an air atmosphere.

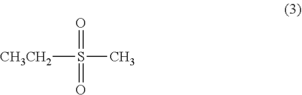

### 1.15 Comparative Example 10

The lithium salt represented by the above chemical formula (1) and the 3-methylsulfolane represented by the following chemical formula (4) were weighed so that the molar ratio of the 3-methylsulfolane to the lithium salt was 1.5. After the 3-methylsulfolane was heated to form a melt, the lithium salt was added thereto, and the mixture was stirred with warming to dissolve the lithium salt. After stirring for 2 hours, by allowing to cool to room temperature in air, to obtain an evaluation sample according to Comparative Example 10. The evaluation sample obtained was a mixture of solid and liquid at room temperature (20° C.) under an air atmosphere.

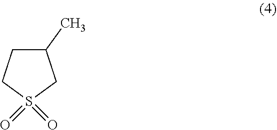

### 1.16 Comparative Example 11

The lithium salt represented by the above chemical formula (1) and the 1,4-butanesultone represented by the following chemical formula (5) were weighed so that the molar ratio of the 1,4-butanesultone to the lithium salt was 2.0. The 1,4-butanesultone was heated to a melt, and the lithium salt was dissolved by adding the lithium salt and stirring with warming. After stirring for 2 hours, and allowed to cool to room temperature to obtain an evaluation sample according to Comparative Example 11. The evaluation sample obtained was a mixture of solid and liquid at room temperature (20° C.) under an air atmosphere.

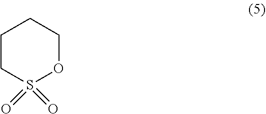

### 1.17 Comparative Example 12

The lithium salt represented by the above chemical formula (1) and the 1,3-propanesultone represented by the following chemical formula (6) were weighed so that the molar ratio of the 1,3-propanesultone to the lithium salt was 2.0. The 1,3-propanesultone was heated to a melt, and the lithium salt was dissolved by adding the lithium salt and stirring with warming. After stirring for 2 hours, by allowing to cool to room temperature in air, to obtain an evaluation sample according to Comparative Example 12. The evaluation sample obtained was a mixture of solid and liquid at room temperature (20° C.) under an air atmosphere.

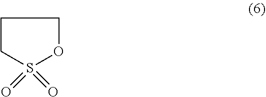

### 2. Ion Conductivity Measurement

Each of the evaluation samples according to Examples 1 to 5 and Comparative Examples 2, 5, and 8, which were solid at room temperature, was put into a press cell and pressed to prepare a blocking cell. For the blocking cell, by performing AC impedance measurement, to determine the resistance value associated with the ion conduction, to calculate the ion conductivity from the cell shape. Measuring conditions are as follows: Temperature 25° C., magnitude 10 mV, and frequency 1M˜10 mHz.

### 3. Crystal Structure Analysis

Each of the evaluation samples according to Examples 1 to 5 and Comparative Examples 2, 5, and 8, which were solid at room temperature, were subjected to X-ray diffraction measurement with CuKα rays using an X-ray diffraction apparatus. The measuring conditions were 2°/min scan speed and 0.01° step-width.

### 4. Evaluation Results

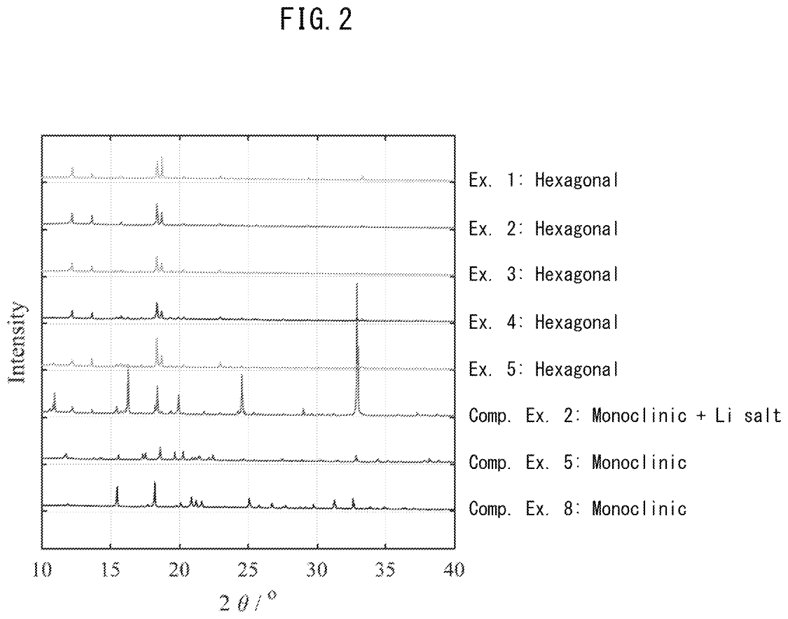

The evaluation results are shown in Table 1 below. Further, FIG. 2 shows the X-ray diffraction pattern of each of the evaluation samples according to Examples 1 to 5, Comparative Examples 2, 5 and 8.

TABLE 1

| | Electron | | | | | |
|---|---|---|---|---|---|---|
| | donating | | | Condition | Ion | |
| | organic | | Molar ratio | at room | conductivity | Crystal |
| | compounds A | Lithium salt B | (A/B) | temperature | S/cm | structure |
| Comparative | Chemical | Chemical | 3.5 | Solid- | — | — |
| Example 1 | formula (2) | formula (1) | | liquid | | |
| Example 1 | Chemical | Chemical | 3.1 | Solid | 7.1E−7 | Hexagonal |
| | formula (2) | formmila (1) | | | | |
| Example 2 | Chemical | Chemical | 3.0 | Solid | 7.7E−7 | Hexagonal |
| | formula (2) | formula (1) | | | | |
| Example 3 | Chemical | Chemical | 2.9 | Solid | 3.0E−6 | Hexagonal |
| | formula (2) | formula (1) | | | | |
| Example 4 | Chemical | Chemical | 2.5 | Solid | 1.4E−6 | Hexagonal |
| | formula (2) | formula (1) | | | | |
| Example 5 | Chemical | Chemical | 2.0 | Solid | 1.0E−6 | Hexagonal |
| | formula (2) | formula (1) | | | | |
| Comparative | Chemical | Chemical | 1.5 | Solid | 2.8E−7 | Monoclinic + |
| Example 2 | formula (2) | formula (1) | | | | Li salt |
| Comparative | Chemical | LiPF₆ | 4.0 | Solid- | — | — |
| Example 3 | formula (2) | | | liquid | | |
| Comparative | Chemical | LiFSA | 1.5 | Solid- | — | — |
| Example 4 | formula (2) | | | liquid | | |
| Comparative | Chemical | LiFSA | 1.0 | Solid | 7.6E−8 | Monoclinic |
| Example 5 | formula (2) | | | | | |
| Comparative | Chemical | LiTFSA | 2.0 | Liquid | — | — |
| Example 6 | formula (2) | | | | | |
| Comparative | Chemical | LiTFSA | 1.5 | Solid- | — | — |
| Example 7 | formula (2) | | | liquid | | |
| Comparative | Chemical | LiTFSA | 1.0 | Solid | 3.8E−9 | Monoclinic |
| Example 8 | formula (2) | | | | | |
| Comparative | Chemical | Chemical | 1.0 | Liquid | — | — |
| Example 9 | formula (3) | formula (1) | | | | |
| Comparative | Chemical | Chemical | 1.5 | Solid- | — | — |
| Example 10 | formula (4) | formula (1) | | liquid | | |
| Comparative | Chemical | Chemical | 2.0 | Solid- | — | — |
| Example 11 | formula (5) | formula (1) | | liquid | | |
| Comparative | Chemical | Chemical | 2.0 | Solid- | — | — |
| Example 12 | formula (6) | formula (1) | | liquid | | |

From the results shown in Table 1 and FIG. 2, it can be seen that:

In the case that sulfolane and a lithium salt represented by Chemical Formula (1) were used, the evaluation sample according to Comparative Example 1 in which the molar ratio of sulfolane to lithium salt was 3.5 did not become a solid single phase and could not be used as a molecular crystal. In contrast, the evaluation samples according to Examples 1 to 5 in which the molar ratio of sulfolane to lithium salt was 2.0 or more and 3.1 or less showed an ion conductivity of more than 7×10⁻⁷ S/cm, and in particular, the evaluation samples according to Examples 3 to 5 in which the molar ratio was 2.0 or more and less than 3.0 (particularly, 2.9 or less) showed an ion conductivity of more than 1.0×10⁻⁶ S/cm. Incidentally, the evaluation sample according to Comparative Example 2 in which the molar ratio was 1.5, as compared with Examples 1 to 5, the ionic conductivity was lowered. In the evaluation sample according to Comparative Example 2, an X-ray diffraction peak derived from a lithium salt raw material was confirmed in addition to a crystal structure of a monoclinic crystal. On the other hand, the evaluation samples according to Examples 1 to 5, which showed high ionic conductivity, consisted of a single phase of hexagonal structure. It is considered that the hexagonal structure has a pass through which Li can diffuse in the x-y plane, and thus, the in-solid ionic conductivity is increased.

The evaluated sample according to Comparative Example 3 using LiPF₆ as a lithium-salt did not become a solid-state single phase at any molar ratio up to a molar ratio of 4.0. When the lithium salt became more concentrated, the lithium salt did not dissolve, and a molecular crystal could not be obtained.

Evaluation samples according to Comparative Examples 4 and 5 using LiFSA as a lithium-salt became a solid single phase at a molar ratio of 1.0, but its crystalline structure was monoclinic, and the ionic conductivity was as low as 10⁻⁸ S/cm order.

Evaluation samples according to Comparative Examples 6 to 8 using LiTFSA as a lithium-salt became a solid single phase at a molar ratio of 1.0, but the crystalline structure was monoclinic, and the ionic conductivity was as low as 10⁻⁸ S/cm order.

The evaluation samples according to Comparative Examples 9 to 12 in which those other than sulfolane were used as the electron-donating organic compound did not become a solid single phase and could not be used as a molecular crystal. It is considered that all of them do not adopt a stable structure as a crystal.

From the above results, it can be said that a molecular crystal comprising the following configurations (A) and (B) has ion conductivity.
(A) The molecular crystal contains sulfolane and a lithium salt represented by the above chemical formula (1).
(B) The molar ratio of said sulfolane to said lithium salt is 2.0 or more and 3.1 or less.

### REFERENCE SIGNS LIST

100 Battery
10 Positive electrode current collector
20 Positive electrode active material layer
30 Electrolyte layer
40 Negative electrode active material layer
50 Negative electrode current collector

1. A molecular crystal, comprising a sulfolane and a lithium salt represented by the following chemical formula (1), wherein
a molar ratio of the sulfolane to the lithium salt is 2.0 or more and 3.1 or less.

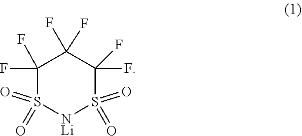

2. The molecular crystal according to claim 1, having a hexagonal structure.

3. The molecular crystal according to claim 1, wherein
the molar ratio of the sulfolane to the lithium salt is 2.0 or more and less than 3.0.

4. An electrochemical device, comprising: an ionic conductor, wherein
the ionic conductor has a molecular crystal according to claim 1.

5. A method for producing a molecular crystal, the method comprising:
mixing sulfolane and a lithium salt represented by the following chemical formula (1) to obtain a solution containing the sulfolane and the lithium salt and having a molar ratio of the sulfolane to the lithium salt of 2.0 or more and 3.1 or less, and
cooling the solution to obtain the molecular crystal. [chemical structure]
